SEQUENCE LISTING (3 sequences)

SEQ ID NO: 1 - protein, 15 aa, Capra hircus
MSSLVRRIISTAKAP

SEQ ID NO: 2 - protein, 15 aa, Capra hircus
NIGEILKAAGCDFTN

SEQ ID NO: 3 - protein, 15 aa, Capra hircus
VVKATVLLADINDFS

(19) United States
(12) Patent Application Publication
(10) Pub. No.: US 2004/0180835 A1
(43) Pub. Date: Sep. 16, 2004

(54) Use of the protein uk114 or of fragments thereof for the treatment and prevention of the endotoxic shock

(76) Inventors: Alberto Bartorelli, Crans sur Sierre (CH); Pierferdinando Nicoletti, Milano (IT); Alberto Panerai, Milano (IT)

Correspondence Address: YOUNG & THOMPSON, 745 SOUTH 23RD STREET 2ND FLOOR, ARLINGTON, VA, 22202

(21) Appl. No.: 10/474,379

(22) PCT Filed: Apr. 9, 2002
(86) PCT No.: PCT/EP02/03933

(30) Foreign Application Priority Data
Apr. 10, 2001 (IT) M 101A000762

(51) Int. Cl. A61K 38/10; C07K 7/08

(52) U.S. Cl. 514/14; 514/15; 530/326; 530/327

(57) ABSTRACT

Use of the protein UK114 or of fragments thereof for the treatment and prevention of the endotoxic shock.

The present invention refers to the use of the UK114 protein or of fragments thereof for the treatment and prevention of the endotoxic shock.

Shock is a clinical syndrome characterised by an insufficient tissutal perfusion and by several clinical symptoms such as anxiety, confusion, coma, hyperventilation, oliguria, anuria, hypotension, vasoconstriction and vasodilation. The symptoms may be so serious to cause fatalities with high frequency (Beal A. et al., JAMA, 1990, 271:226-233).

Sepsis is in order of frequency the third cause of shock after haemmorrages and myocardial infarct. In particular, the gram-negative bacteria are the main cause of the septic shock even though infections by fungi, ricketsiae and viruses may also play a role (Beal A. et al., JAMA, 1990, 271:226-233).

The onset of endotoxic shock in healthy subjects less than 40 years old is rare but its frequency may be increased by diabetes, endoscopic operations, chronic hepatopathies, blood dyscrasia, and by the prolonged use of immunosuppressive drugs such as corticosteroids and cyclosporin A.

Until now, there is no therapeutic approach for the treatment of endotoxic shock. This implies a very high death risk in hospitalised patients, particularly in case of gram positive infections, approaching about 50-80% (Beal A. et al., JAMA, 1990, 271:226-233).

It has now been found that the protein UK114, disclosed in WO 96/02567 and in WO 00/63368, herein incorporated by reference, up to now studied for a series of therapeutic applications comprising the treatment of neoplasias (WO 96/02567), autoimmune diseases (WO 98/11909), TNF-induced diseases (WO 98/42366), AIDS (WO 98/11137), treatment and prevention of transplanted organs (WO 00/78329), may be advantageously used in the treatment of endotoxic shock.

It has also been found that the same pharmacological activity is shared by peptides having from 10 to 20 amino acids, preferably from 12 to 18 amino acids, having sequences corresponding to the 1-20 sequence (N-terminal) and to the 55-95 sequence of the native UK114 protein.

The invention refers therefore also to said peptides and to the pharmaceutical compositions containing them.

The preferred peptides of the invention have the following sequences, corresponding respectively to the 1-15, 61-75 and 76-90 of the native protein. Met-Ser-Ser-Leu-Val-Arg-Arg-Ile-Ile-Ser-Thr-Ala-Lis-Ala-Pro (Sequence Id 1, peptide 1-15) Asn-Ile-Gly-Glu-Ile-Leu-Lys-Ala-Ala-Gly-Cys-Asp-Phe-The-Asn (Sequence Id 2, peptide 61-75) Val-Val-Lys-Ala-Thr-Val-Leu-Leu-Ala-Asp-Ile-Asn-Asp-Phe-Ser (Sequence Id 3, peptide 76-90).

The peptide corresponding to the sequence 76-90 is particularly preferred.

The invention also comprises peptides functionally equivalent to the peptides defined above.

By the term “functionally equivalent peptide”, it is meant a peptide having a comparable activity to that of the unmodified peptide and which, in comparison to said sequences, has conservative amino acid substitutions, and/or deletions and/or insertions and/or substitutions with corresponding amino acids of the D series and/or derivatised at the amino, hydroxy and thio groups and/or retro-inverted amino acids.

Said modifications are within the skill of any expert technician and may yield more stable peptides against the enzymatic hydrolysis and/or having different pharmacokinetics characteristics. The preparation of the peptides is carried out with conventional methods, preferably using the solid-phase synthesis according to Merrifield or similar methods.

For the considered therapeutic uses, the UK 114 protein and the corresponding peptides will be administered parenterally, in form of suitable pharmaceutical compositions, at dosages that may be easily determined by the clinicians according to the pharmacokinetics and toxicological characteristics of the UK 114 protein or of the used peptide, as well as according to the severity of the disease and to the patient's conditions (weight and age). The daily dosages will usually range from 0.1 to 10 mg of UK 114 or peptide, by the intramuscular or subcutaneous route, optionally divided in more administrations.

The pharmacological activity of UK 114 and of the peptides of the invention has been studied on the murine model of LPS-induced shock, as reported in the following Example.

### EXAMPLE

Balb/c mice 6-8 weeks old were treated i.p. with 30 mcg of UK114 in 100 μl PBS (group B) or with 4 mcg of peptide 76-90 (group C) 24 hours and one hour before the administration of 500 mcg of LPS. A control group of mice (group A) was tretated with 100 μl PBS according to the same protocol used for groups C and B.

A further group was treated only with UK114 one hour after the administration of LPS (group D). This experimental approach more closely resembles the clinical practice where it is difficult to intervene prophylactically.

The death rate of the animals in each group was recorded every 12 hours up to the third day from the LPS injection.

As shown in the Table, the i.p. administration of LPS caused the death in most control animals (treated with PBS) within the observation period (72 hours post LPS).The animals prophylactically treated with UK 114 or with the peptide 76-90 were clearly protected from the lethal effects of LPS, and the death rate was significantly lower than in the control group. This protective effect, even though less marked, was also present in the mice therapeutically treated with UK 114 only 1 hour after the LPS administration. The protective effect of UK 114 was not simply transient since none of the survived mice died in the subsequent follow-up month.

These data show that the administration of UK114 not only can prevent but also “cure” the lethal effects of the LPS injection in a known animal model of endototoxic shock. The obtained data are strongly suggestive for an important role of UK 114 in the prevention of endotoxic shock in subjects at risk and in the treatment of the subjects already affected by endotoxic shock.

TABLE
Effects of the treatment with UK 114 and with the peptide
76-90 on LPS-induced endotoxic shock in mice.

| Group (n) | Treatment | Frequency and time* | Death-rate (at 72 h) |
|---|---|---|---|
| A (50) | PBS | −24, −1 | 40/50(80%) |
| B (50) | UK114 | −24, −1 | 14/50(28%)** |
| C (50) | pep. 76-90 | −24, −1 | 25/50(50%)*** |
| D (50) | UK114 | +1 | 25/50(50%)*** |

*hours from the LPS administration
**p < 0.0001 vs group A
***p = 0.003 vs group a by statistical analysis with Chi-square.

1. Use of the UK 114 protein or of fragments thereof for the treatment and the prevention of endotoxic shock.

2. Peptides having from 10 to 20 amino acids, having sequences corresponding to the 1-20 sequence (N-terminal) and to the 55-95 sequence of the native UK114 protein.

3. Peptides according to claim 2 having from 12 to 18 amino acids.

4. Peptides according to claim 3, having sequence Id 1, Id 2 or Id 3.

5. Peptides of claims 2-4 modified by conservative amino acid substitutions, and/or deletions and/or insertions and/or substitutions with corresponding amino acids of the D series and/or derivatised at the amino, hydroxy and thio groups and/or retro-inverted amino acid.

6. Pharmaceutical compositions comprising a peptide of claims 2-5 in admixture with a suitable carrier.
